(19) United States
(12) Patent Application Publication
(10) Pub. No.: US 2022/0397545 A1
(43) Pub. Date: Dec. 15, 2022

(54) PHOTOCHEMICAL-BASED METHOD FOR ENZYME IMMOBILIZATION ON BIOSENSOR ELECTRODES

(71) Applicant: LAXMI THERAPEUTIC DEVICES, INC., Goleta, CA (US)

(72) Inventors: Leanne Beer, San Francisco, CA (US); Isabella Camille Darke, Boston, MA (US); Ashley Nicole Farnkopf, Santa Barbara, CA (US); William Peter Van Antwerp, Santa Clarita, CA (US)

(21) Appl. No.: 17/348,465

(22) Filed: Jun. 15, 2021

(51) Int. Cl. G01N 27/327

(52) U.S. Cl. CPC G01N 27/3272

(57) ABSTRACT

A method for forming an enzymatic biosensor includes preparing an aqueous solution including an enzyme and photocurable components, depositing the aqueous solution on a surface of a working electrode of a substrate, illuminating the working electrode with ultraviolet (UV) light to cure the aqueous solution, and crosslinking the enzyme deposited on the working electrode with solution phase or vapor phase crosslinking after curing the aqueous solution.

### BACKGROUND

### Field

The present disclosure generally concerns medical devices and tools used for measuring analytes in the body. More specifically, embodiments of the invention relate to a method for immobilizing and stabilizing an enzyme matrix on an electrode of a medical device used for measuring analytes in the body, such as at a working electrode of an electrochemical biosensor.

### Description of Related Art

Recent years have seen increased interest in wearable sensors for measuring analytes in the body (e.g., glucose levels), because such sensors might greatly ease analyte measurement. In particular, there has been growing interest for wearable sensors that selectively measure analytes that must be continuously monitored or at least frequently monitored. For example, monitoring glucose levels is particularly important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications. Other analytes that can use this same type of enzyme-based technology include lactate (athletes, sepsis), cholesterol, creatinine (CKD), hydroxybutyrate (ketones), urease (kidney diseases), bilirubin, and many others.

Traditionally, glucose has been monitored in capillary blood, typically from a fingertip. A user of these traditional glucose measuring devices would prick a fingertip with a blood lancet to produce a drop of blood. The user would place the drop of blood on a test strip after inserting the test strip into the meter; the meter would then measure the glucose concentration in the blood. Although widely used, traditional glucose monitoring devices are limited because they can only measure blood glucose levels at a single point in time. This can be problematic because blood glucose levels can fluctuate throughout a day, especially after food is digested. Regular monitoring of blood glucose levels may require multiple finger prick measurements, which may be prone to some technique errors and/or other variations based on inconsistent user and external variables. In addition, frequent blood pricks can be painful or otherwise overly intrusive, and users may be hesitant to take a sufficiently appropriate number of blood glucose measurements to get any sort of glucose rate of change data. Rate of change data is particularly important for patients dosing insulin since a proper meal bolus needs to be adjusted for the current glucose rate of change. For example, if a patient eats a 100 g carbohydrate meal that would need 6 units of insulin based on her insulin sensitivity, the correct dose would be 5.7 U if the BG is going down at more than 1.5 mg/dl-min and would be 6.2 or 6.3 U if the BG is rising. Only CGM systems can give the patient that type of information.

### SUMMARY

In contrast to traditional glucose monitors, wearable devices can allow for continuous glucose monitoring (e.g., continuous glucose monitors (CGM)). CGMs are always active and continuously measure glucose levels at set intervals (e.g., measured every ten seconds and readings reported every five minutes). The continuous data can allow users to track glucose levels over the course of a day or over a few days and help users make more informed decisions about their diet, physical activity, and medications. CGMs operate by inserting a small sensor under the skin, typically on the abdomen or the back of the upper arm, although sensors have been worn on other parts of the body including the thighs, lower back, and buttocks. FIGS. 1A and 1B show examples of wearable devices that are worn on a human body, where in FIG. 1A a CGM 1 is worn or otherwise attached to the abdomen or torso region of a patient or subject, and in FIG. 1B the CGM 1 is alternatively worn or otherwise attached to an upper arm of a patient or subject. Other sensors may also be worn on other parts of the body. The sensors can be held in place by an adhesive and can wirelessly transmit glucose measurements to, for example, a separate monitoring device. However, unlike finger stick capillary blood devices, CGMs instead measure glucose in interstitial fluid (e.g., glucose in the fluid between cells) rather than glucose in blood directly.

An example of a wearable CGM is schematically shown in FIGS. 2A to 3. FIG. 2A shows a perspective view of an embodiment of a wearable CGM 1, FIG. 2B shows a side view of the wearable CGM 1, and FIG. 2C shows a bottom view of the wearable CGM 1. The CGM 1 may generally include a body or housing 10 having a top end 11 and a bottom end 12. An adhesive 13 may be found on the bottom end, and may be in the form of, for example, a tape that has a perimeter that extends slightly outside of the perimeter of the housing 10, to improve adhesion to the patient's body. At the bottom end 12, there may also be a hole or opening 14, through which a needle sensor 15 extends. The needle sensor 15 may only protrude slightly out of the bottom end 12 and may be connected in the housing to another circuitry. FIG. 3 shows a schematic block diagram of the CGM 1. The needle sensor 15 may be connected to a PCB 16 or another circuitry. The PCB 16 may further include or be otherwise connected to a transmitter/receiver 17, a controller 18, and a battery 19. The transmitter/receiver 17 allows the CGM 1 to communicate with a separate monitoring device, the controller 18 can control functionality and monitoring of the CGM 1, and the battery 19 can supply power to the components of the CGM 1.

The above analyte measurement methods, including monitoring with CGMs, are still categorized as invasive, that is, they require entry into the body or a body cavity (e.g., where a probe is used to percutaneously access the body fluid being analyzed). In contrast, non-invasive glucose monitoring methods have also been explored but have proven difficult to implement for various reasons such as reduced accuracy. Meanwhile, invasive methods have traditionally used a probe in the form of a thin flexible sensor inserted into the lipid layer of the skin approximately 6-9 mm below the surface. Historically, there has been more limited interest in rigid needle-based wearable analyte sensors because of recognized difficulties in designing and manufacturing a sufficiently compact and reliable needle-based system. To address this and similar issues, needles can, for example, be integrated with electrodes that can be used for various applications, including electrochemical sensing and electrical stimulation.

In addition, for example as seen in embodiments of the invention, needles can also be integrated with enzyme matrices. An enzyme is a catalyst that can speed up the rate of a chemical reaction without itself being altered in the chemical reaction. The composition of the enzyme matrix can depend on the analyte being measured. For example, CGMs may have sensors that incorporate glucose oxidase (GOx) enzyme matrices. GOx is an enzyme that catalyzes the oxidation of glucose into hydrogen peroxide (H₂O₂) and gluconolactone (C₆H₁₀O₆). The chemical formula for the oxidation of glucose is provided below:

$${C}_{6} {H}_{12} {O}_{6} + {O}_{2} {⟶}^{GOx} {H}_{2} {O}_{2} + {C}_{6} {H}_{10} {O}_{6}$$

Using this formula, CGMs calculate blood glucose levels by measuring the amount of hydrogen peroxide produced by the oxidation of glucose. To this end, the CGM sensors can, for example, have a working electrode that uses a fixed potential electrochemical measurement technique where the hydrogen peroxide is oxidized (e.g., undergoes an oxidation reaction) and the electrical current produced is measured as the sensor signal. Based on measurement of the electrical current, the CGM can determine the concentration of glucose.

An example of a needle sensor for a CGM is schematically shown in FIGS. 4A-4C. FIG. 4A shows a perspective view of a needle sensor 15, FIG. 4B shows a top view of the needle sensor 15, and FIG. 4C shows a close-up view of a tip of the needle sensor 15 including the sensor portion. The needle sensor 15 includes a base 151, a shaft 152, and the needle tip 153. The needle tip 153 may include various electrodes 154a, 154b, and 154c that facilitate the glucose monitoring. The electrodes 154a, 154b, 154c are electrically connected to corresponding larger contacts 155a, 155b, 155c at the base, for easy connectivity with the PCB 16 of FIG. 3.

In CGMs, enzyme systems may be functionalized on a sensor by a number of methods, including submerging the sensor into a deposition solution during manufacturing or drop coating the enzyme matrix onto the sensor surface. The deposition solution may include a mixture of components, including a particular concentration of the desired enzyme and a protein. In both of these cases, the enzyme in the deposition solution can form a layer on the needle surface via intermolecular interactions. However, the enzyme layer may form over undesired parts of the sensor producing hydrogen peroxide which does not contribute to the signal. This non-specific hydrogen peroxide may diffuse into the tissue in vivo leading to potential tissue irritation.

Additionally, CGM sensors that use GOx and measure hydrogen peroxide will have an oxygen deficit problem. In the glucose oxidation reaction, one mole of oxygen is needed for one mole of glucose. In the body tissue, oxygen levels are typically 20 μM while tissue glucose concentration can be as high as 28 mM (500 mg/dl).

Moreover, problems particularly arise with cost-effective manufacturing of small and structurally robust needles with integrated electrodes. Very small needles (e.g., microneedles) are desired to reduce sensor size and minimize user sensation (e.g. pain or discomfort) during needle insertion, but traditional methods of manufacturing microneedles produce needles of silicon (via conventional silicon micromachining or use of structural lithography resists, e.g., SU-8 photoresists), or plastic needles (via micro molding methods). Silicon microneedle arrays are limited in length by the thickness of available silicon wafers (a wafer is no more than 750 microns thick) and thus are limited to 400 or so microns in length which are not long enough to reach the true dermis. Molded plastic microneedle arrays are not mechanically strong enough at lengths that are in the 1-1.5-millimeter range.

The exemplary embodiments of the present invention disclosed herein are directed to an electrochemical method for enzyme immobilization on biosensor electrodes.

Embodiments of the present disclosure include a method for immobilizing and stabilizing an enzyme matrix on a working electrode of an electrochemical biosensor. In some embodiments, the working electrode of the electrochemical biosensor can be used to measure analytes in the body (e.g., glucose levels).

According to various embodiments of the present disclosure, the method incorporates complementary electrochemical techniques that can deposit an inner enzymatic sensing layer and an outer polymer membrane in a highly selective and easily controllable manner. The techniques for depositing an enzyme matrix include constant voltage, constant current DC, AC with unbalanced positive and negative voltages as well as a sequence of pulses of various durations and voltages. In general, enzymes and other proteins have an isoelectric point (pI) in the pH 4-5 range. Glucose oxidase has a pI of 4.2. This means that at pH 7, the enzyme is predominantly negatively charged. At pH 3.5 the enzyme is predominantly positively charged. For electro-osmotic delivery, both low and high pH matrices can be used. At pH 7.4 a positive charge on the working electrode will pull enzyme to the electrode, while at pH 3.5 a negative charge will do the same. In some preferred embodiments, the complementary electrochemical techniques can include galvanostatic adsorption of an enzyme matrix and electropolymerization of membrane via cyclic voltammetry.

The galvanostatic adsorption technique involves holding a constant current at an electrode while the electrode is immersed in an electrolyte which contains the species to be absorbed on the electrode (e.g., a deposition solution). The polarization of the electrode can be controlled by maintaining the electrode at a constant current. Based on the polarization of the electrode, certain compounds can be attracted (e.g., drawn by electrostatic forces) to the surface of the electrode. In some embodiments, the polarization of the electrode can be adjusted to attract enzymes in the deposition solution to the electrode surface. The electrostatic forces can hold the enzyme at the surface of the electrode. It is well understood that other approaches can also be used as described in herein, such as unbalanced AC deposition, constant voltage deposition and pulse sequence deposition.

The electrochemical polymerization technique is a process for creating polymer films on an electrode by controlling the number of electric potential cycles or current that is applied to the electrode while immersed in an electrolyte containing monomeric species (e.g., a monomer that can be polymerized by applying an appropriate voltage/current, such monomers are electro-polymerizable). As with the deposition of the enzyme, several electrochemical approaches can be used to create the electropolymerized polymers. These may include constant voltage, constant current, cyclic voltammetry, unbalanced AC as well as a series of pulses with constant or varying voltages and times. Electrodeposited polymers can be either conducting or non-conducting, and the polymer system is chosen for specific analytical reasons. Some examples of monomers that can be electropolymerized to generate conducting polymers are pyrrole, thiophene, indole, carbazole, triphenylene and many of their derivatives. Some non-conducting electro-polymers are those made from phenol and phenol derivatives, phenylene diamines, chlorophenyl amide, and tyramine. Tyramine is one of the most interesting since it has a free amino group that can be used to covalently crosslink the enzyme to the resulting polymer.

In some embodiments, cyclic voltammetry (CV) can be used to create the polymer layer on the electrode. In cyclic voltammetry, the potential of the working electrode is scanned over a specific range and cycled such that the resulting current can be measured. For example, a potential scan can begin from a greater potential and end at a lower potential. The scan can then cycle from the lower potential back to the greater potential. The current created by the cycle changes can create a polymer film on the surface of the working electrode due to oxidation or reduction of the monomeric species in the deposition solution for this process.

Enzymes after deposition need to be immobilized to preserve their enzymatic activity. Immobilization is typically done by crosslinking the enzyme either to itself, to another protein such as albumin, or by covalent attachment to the electropolymerized polymer overlayer. There are multiple methods for crosslinking of the enzyme including glutaraldehyde and other di-aldehydes, carbo-diimides of varying chain lengths and a wide variety of heterobifunctional reagents with two different bonding domains.

The enzyme immobilization method can be used in various enzymatic sensing systems, including, but not limited to, sensing systems that measure lactate, alcohol, or glucose. In some embodiments, the method can be used for immobilizing and stabilizing an enzyme matrix on a glucose sensor. Glucose oxidase (GOx) is one possible enzymatic species that can be used with the glucose sensor but other enzymatic species, such as glucose dehydrogenase, may also be incorporated.

According to one embodiment, GOx can be adsorbed on the surface of the glucose sensor using galvanostatic adsorption. In the galvanostatic adsorption step, a current can pass through the working electrode to create an electrical charge that attracts the negatively charged GOx dissolved in a solution with pH higher than the isoelectric point to the surface of the working electrode. Alternatively, the GOx can be driven to the electrode with a negative voltage if the GOx is dissolved in a solution where the pH is below the pI. The galvanostatic attraction increases the amount of enzyme coated on the sensor, while maintaining a controlled deposition at the working electrode only. In some embodiments, a crosslinking partner (e.g., another protein (e.g., stabilizing protein) that can be used as a crosslinking partner to increase enzyme stability) can be included. In some embodiments, human serum albumin (HSA) can be used as a stabilizing protein for the GOx after crosslinking but other stabilizing proteins such as silk fibroin may be incorporated.

After the initial adsorption of GOx, cyclic voltammetry (CV) as well as fixed potential electropolymerization can be used to form a polymer membrane over the adsorbed GOx on the electrode surface. In some embodiments, the polymer layer can be a polytramine membrane however other polymeric systems can be used (e.g., polydopamine, m-phenylenediamine, o-phenylenediamine, polypyrrole). During the electropolymerization, a polymer film encapsulates the previously deposited enzyme, which provides stability and prevents the enzyme from diffusing away from the electrode surface. In some embodiments, the polymer layer can be glucose limiting (e.g., limit the amount of glucose that can pass through the polymer layer) and it can also limit the transport of interfering species into the sensor (e.g., ascorbic acid, acetaminophen).

The galvanostatic adsorption and the cyclic voltammetry electropolymerization steps are relatively short (e.g., on the order of minutes/tens of minutes) and can be expanded to fabricate many devices in parallel. Also, the transition to using cyclic voltammetry instead of potentiostatic or galvanostatic techniques can result in higher yielding, more efficient, and repeatable depositions. This method also avoids the issues that arise with holding low currents and not reaching the necessary oxidation potentials.

Additionally, glutaraldehyde or other chemical induced crosslinking can also be applied to the glucose sensor to impart additional stability to the enzyme system. Glutaraldehyde can be utilized in the vapor or liquid state. In some embodiments, the crosslinking step could be added prior to CV electropolymerization or fixed potential electropolymerization of the polymer, to provide stability to the enzyme matrix by crosslinking the enzyme to stabilizers such as human serum albumin (HSA) prior to addition of polymer membrane. In other embodiments, the glutaraldehyde crosslinking step can be added after the addition of membrane. Using tyramine as an example, the pendant amine groups on the polymer backbone can crosslink directly to the enzyme matrix imparting additional stability to the system.

The electrochemical method for immobilizing the enzyme on the biosensor electrodes can be applied on various electrode types, including, different types of platinum (Pt) including, but not limited to, platinum wires, evaporated titanium platinum (TiPt), Pt black, and screen-printed Pt, as well as other electrode materials including carbon, iridium oxide, microtextured Pt, and a wide variety of nanoparticle based working electrodes.

The electrochemical method for immobilizing the enzyme on the biosensor electrodes disclosed herein provides various advantages. For example, the method is easily scalable and allows for controlled, repeatable deposition of enzymes within the confines of a working electrode. This method also conserves materials and prevents non-specific GOx adsorption onto other parts of the sensor, which mitigates any unnecessary hydrogen peroxide formation which could irritate the tissue in vivo. Additionally, the resulting polymer films can suppress potential interferents, such as acetaminophen or ascorbic acid, and have the potential to be naturally glucose-limiting.

The exemplary embodiments of the present invention disclosed herein are also directed to a photochemical method for enzyme immobilization on biosensor electrodes.

According to various embodiments of the present disclosure, the method incorporates photochemical techniques that can deposit an inner enzymatic sensing layer and an outer polymer membrane in a highly selective and easily controllable manner. In some embodiments, combinations of photochemical techniques and electrochemical techniques can be used to deposit the inner enzymatic sensing layer and the outer polymer membrane in a highly selective and easily controllable manner.

### BRIEF DESCRIPTION OF THE DRAWINGS

FIGS. 1A and 1B schematically show a wearable continuous glucose monitor (CGM) being worn on a patient's torso and on a patient's arm, respectively.

FIG. 2A-2C respectively show a perspective view, a side view, and a bottom view of an embodiment of a CGM according to an embodiment of the invention.

FIG. 3 shows a schematic block diagram of the CGM of FIG. 2.

FIGS. 4A-4C respectively show a perspective view, a top view, and an enlarged view of a tip of a needle sensor of the CGM of FIGS. 2A-3.

FIG. 5 is a flowchart showing a method for immobilizing an enzyme and diffusion limiting polymer on a working electrode of a biosensor needle tip, in accordance with example embodiments of the invention.

FIG. 6A shows the glucose response of a sensor that has been fabricated according to the method disclosed in FIG. 5 using a Pt black working electrode.

FIG. 6B shows the linear response of the sensor under the test conditions described in FIG. 5, where the working electrode is Pt black.

FIGS. 7A and 7B are additional examples of a linear response to glucose for sensors made according to a modified version of the method disclosed in FIG. 5, where the deposition solution also contains HSA stabilizer.

FIG. 8 is a flowchart showing a method for immobilizing an enzyme and diffusion limiting polymer on a working electrode of a biosensor needle tip, which includes stabilizing the enzyme matrix with vapor phase glutaraldehyde crosslinking in accordance with example embodiments of the invention

FIG. 9A shows the deposition trace for the galvanostatic absorption of enzyme onto the working electrode according to the method disclosed in FIG. 5.

FIG. 9B shows the cyclic voltammetry trace for the deposition of the diffusion limiting membrane which encapsulates the enzyme according to the method disclosed in FIG. 5.

FIGS. 9C and 9D are examples of a linear response to glucose for sensors made according to the method disclosed in FIG. 8, where the devices are crosslinked.

FIG. 10A shows the deposition trace for the galvanostatic absorption of enzyme onto the working electrode according to the method disclosed in FIG. 5.

FIG. 10B shows the cyclic voltammetry trace for the deposition of the diffusion limiting membrane which encapsulates the enzyme according to the method disclosed in FIG. 5.

FIG. 10C shows the glucose response of a set of sensors that has been fabricated according to the method disclosed in FIG. 8.

FIG. 11 is a flowchart showing a method for immobilizing an enzyme on a working electrode of a biosensor needle tip using photochemical techniques, in accordance with example embodiments of the invention.

FIG. 12 is a flowchart showing a method for immobilizing an enzyme on a working electrode of a biosensor needle tip using a functionalized enzyme, in accordance with example embodiments of the invention.

FIG. 13 is a flowchart showing a method for immobilizing an enzyme on a working electrode of a biosensor needle tip and adding an electropolymerized membrane over the absorbed enzyme, in accordance with example embodiments of the invention.

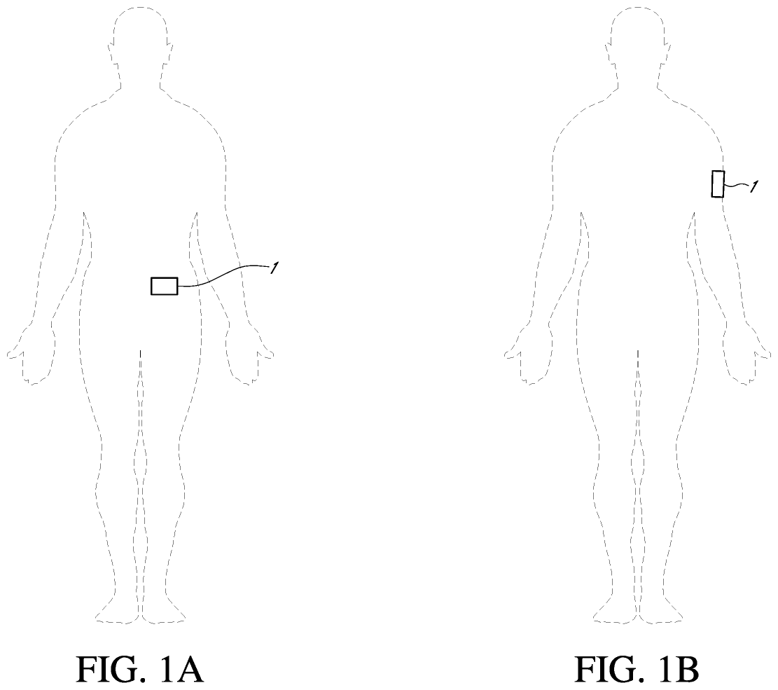

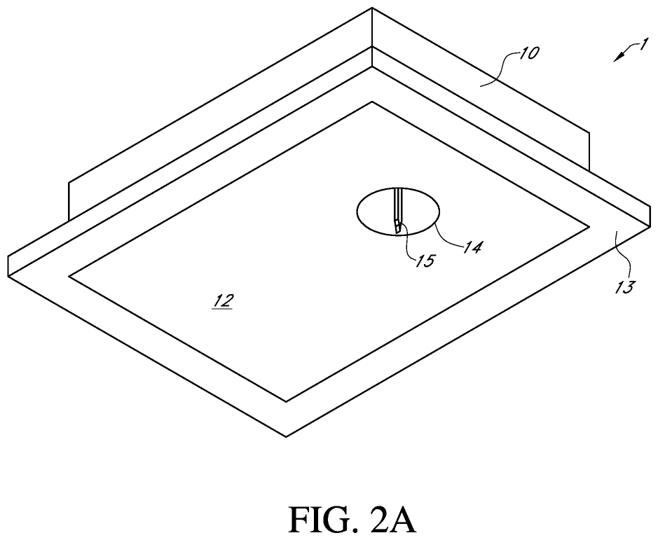

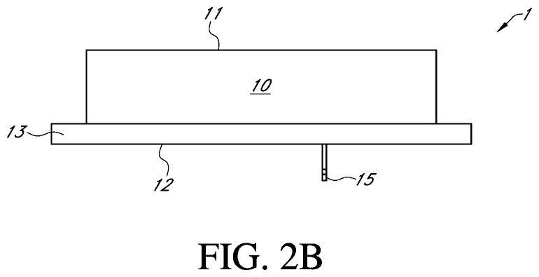

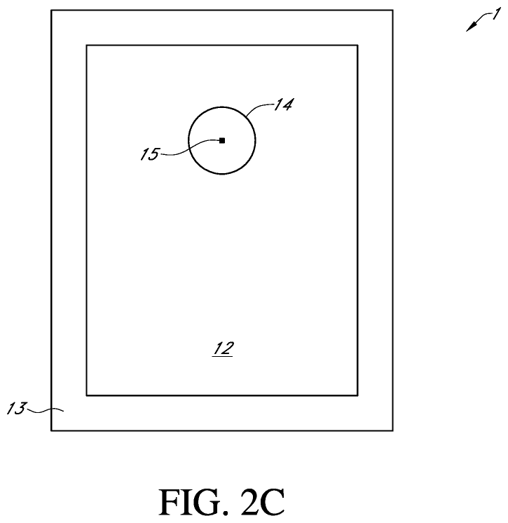

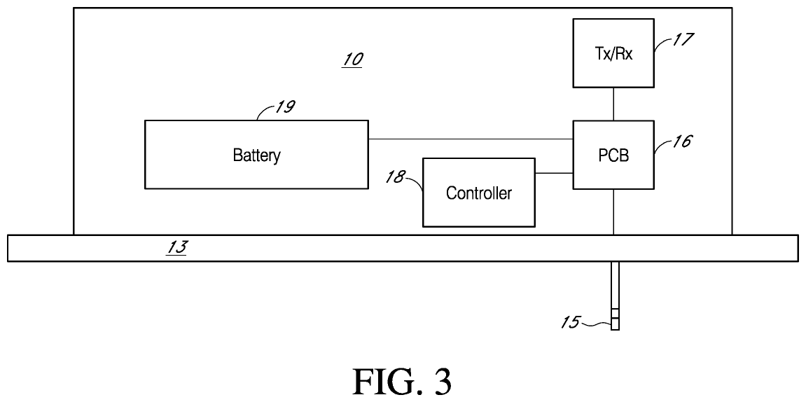

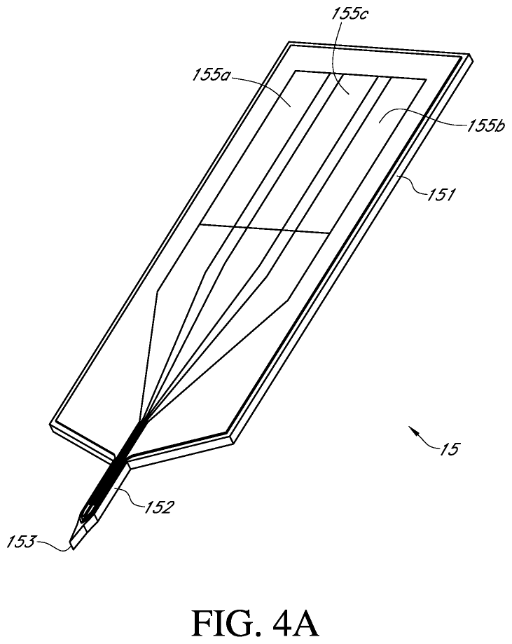

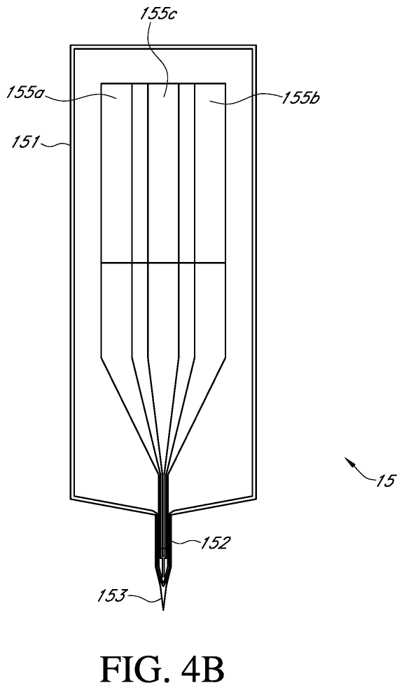

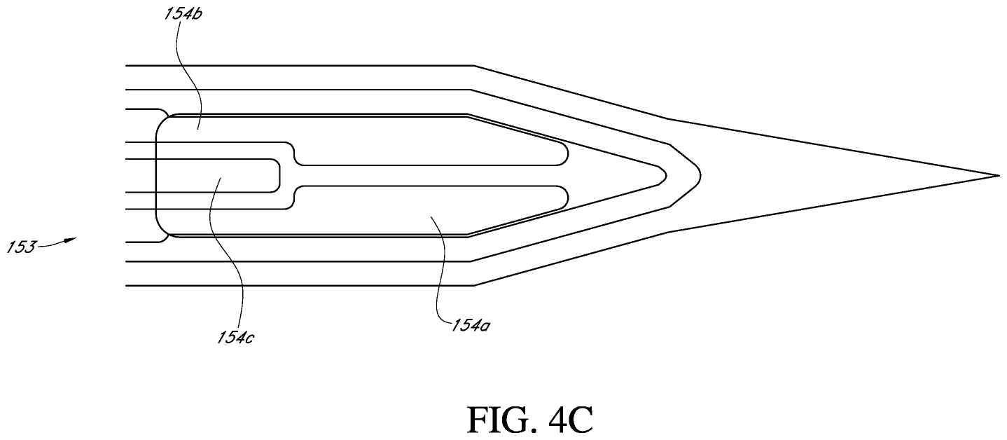

### DETAILED DESCRIPTION

Many previous wearable sensors required piercing of a patient's skin using a rigid needle, with a separate typically soft or flexible, biosensor housed inside the needle, where for example, the needle pierces through the skin with the biosensor inside, followed by removal of the needle to leave the biosensor in place under the skin. By forming a biosensor directly on a tip of a needle according to embodiments of the invention, the same needle can be used to pierce the skin, and can remain under the skin for sensing purposes, eliminating the requirement to manually remove the needle, and thereby reducing the steps needed by an end user to apply the wearable sensor, and potentially also reducing the likelihood of errors during application.

A needle sensor, e.g., where a sensor is integrated or otherwise formed directly on a rigid needle structure, can be accomplished in a variety of ways according to embodiments of the invention. Under one approach, a rigid needle substrate (e.g., a biosensor substrate formed as a needle) including, for example, a biocompatible metal, is first provided, and then a first insulating layer is applied on the substrate. In some embodiments, the insulating layer can then be metalized, or covered with a layer of metal. The sensor circuitry of the needle sensor can then be established using, for example, photolithographic techniques. Then, a second insulating layer may then be formed on the needle sensor to insulate the conductive traces of the circuitry formed on the needle, where for example, only the sensor electrodes and the electrical contacts remain exposed. Some nonlimiting examples of materials that can be used for the rigid substrate or other parts of such a needle sensor are stainless steel, titanium, and ceramics such as alumina or silica, or other non-metals. In addition, the various parts of the needle sensor can further be made of the same or of different materials. Other embodiments of forming a general structure of a general needle sensor that serves the dual purpose of skin puncturing and sensing may include more or less steps than those described above without departing from the scope of the invention. In other embodiments, the biosensors according to embodiments of the invention can also still be constructed on a more flexible substrate instead of on a more rigid substrate.

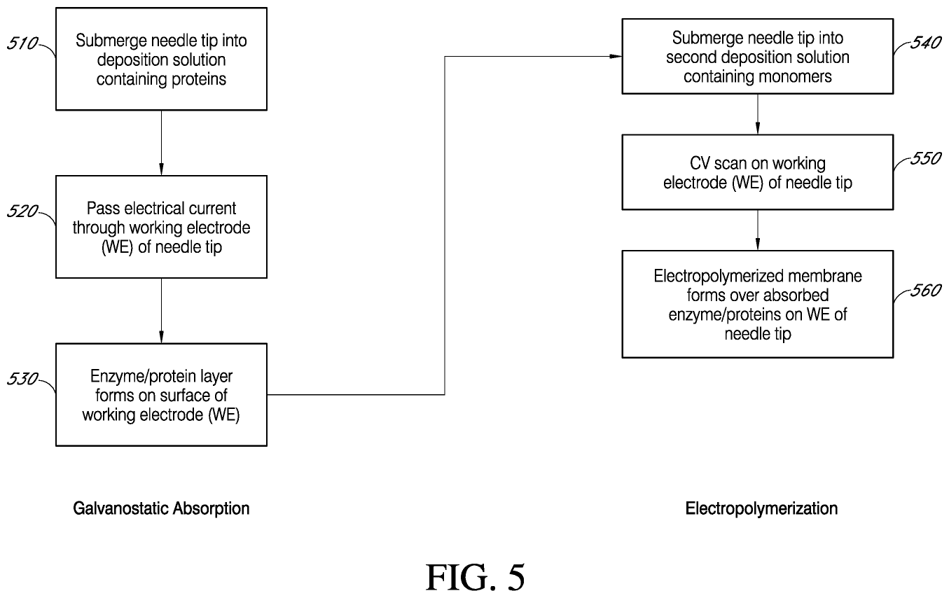

More detailed steps for forming a workable biosensor on a needle tip will now be described. FIG. 5 is a flowchart showing a method for immobilizing an enzyme and diffusion limiting polymer on a working electrode of a biosensor needle tip, in accordance with example embodiments of the invention. According to some example embodiments, the number and order of operations illustrated in FIG. 5 may vary. For example, according to some example embodiments, there may be fewer or additional operations, unless otherwise stated or implied to the contrary.

Referring to FIG. 5, the glucose oxidase (GOx) can be absorbed on the surface of the platinum black working electrode (WE) using galvanostatic adsorption. According to the galvanostatic adsorption technique, the needle sensor can be submerged into a deposition solution containing proteins in step 510. An external counter electrode (CE) and/or an external reference electrode (RE) can also be placed in the deposition solution. In some embodiments, the deposition solution can be composed of 4% wt/vol GOx in phosphate buffered saline (PBS). While the needle sensor is submerged in the deposition solution, an electrical current can be passed through the platinum black WE in step 520, for example, by utilizing the external CE and/or the external RE. In some embodiments, the current can be set at 1 μA and be held between 50 and 60 seconds. In other embodiments, the current may be held up to 120 seconds. The electric current polarizes the platinum black WE, which attracts the GOx in the deposition solution to the electrode surface. The electrostatic forces can hold the GOx and any stabilizing proteins at the surface of the WE in step 530.

Subsequently, a polymer membrane can be formed over the GOx and associated proteins on the surface of the WE using cyclic voltammetry or fixed potential (CV/FP) electropolymerization. This process involves removing the needle sensor from the initial deposition solution and submerging it into a different container having a second deposition solution containing monomers (e.g., electro-polymerizable monomers) in step 540. The external CE and/or the external RE can also be placed in the second deposition solution. In some embodiments, the second deposition solution can be composed of 5 mM tyramine in PBS. In step 550, CV can be used to reversibly scan the potential of the platinum black WE, for example, by utilizing the external CE and/or the external RE. In some embodiments, the range of the CV scan can be 0 to 1.4V at a scan rate of 10 mV/sec for 1 cycle. The CV scan can result in a polytramine membrane deposited over the absorbed GOx on the platinum black WE in step 560. The polytramine membrane may be glucose limiting.

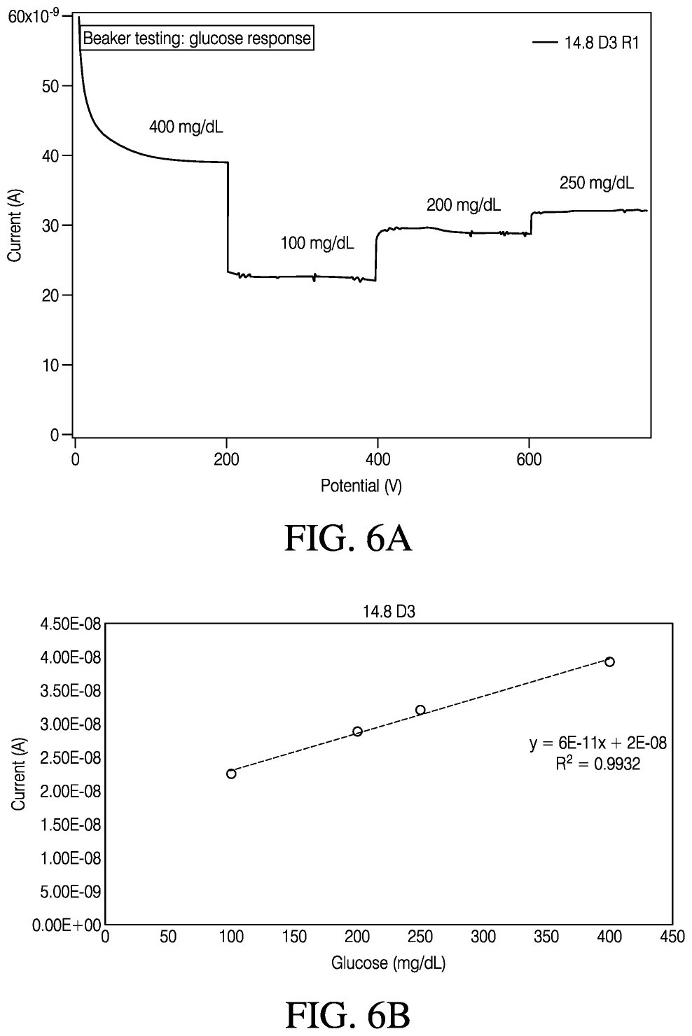

FIG. 6A shows the glucose response of a sensor that has been fabricated according to the method disclosed in FIG. 5. FIG. 6B shows the linear response of the sensor under these test conditions. In FIGS. 6A and 6B, the working electrode is Pt black.

In an alternative method similar to that shown in the method of FIG. 5, a stabilizing protein, such as human serum albumin (HSA), can be added to the deposition solution for the galvanostatic adsorption step on the platinum black WE. As with the method described above, according to some example embodiments, the number and order of operations may vary. For example, according to some example embodiments, there may be fewer or additional operations, unless otherwise stated or implied to the contrary.

In this alternative method, HSA can be incorporated into the deposition solution with GOx during the galvanostatic absorption step onto the platinum black WE according to some embodiments. The HSA stabilizes the GOx on the electrode surface after crosslinking, as the glutaraldehyde crosslinks the GOx proteins to the HSA and the HSA prevents GOx-GOx crosslinking which can destabilize the GOx. The GOx/HSA deposition solution can be composed of 4% wt/vol GOx and 4% wt/vol HSA in PBS. Similar to the method described previously, the needle sensor can be submerged in the GOx/HSA deposition solution with an external CE and/or an external RE. While the needle sensor is submerged in the deposition solution, an electrical current can be passed through the platinum black working electrode (WE) on the needle sensor, for example, by utilizing the external CE and/or the external RE. In some embodiments, the current can be set at 1 μA and be held between 50 and 60 seconds. In other embodiments, the current may be held up to 120 seconds. The electric current polarizes the platinum black WE, which attracts the GOx and HSA in the deposition solution to the WE surface. The electrostatic forces can hold the GOx and HSA at the surface of the platinum black WE.

Subsequently, a polymer membrane can be formed over the GOx on the surface of the platinum black WE using cyclic voltammetry (CV) electropolymerization. This process involves removing the needle sensor from the GOx/HSA deposition solution and submerging it into a different container having a second deposition solution containing monomers. The external CE and/or the external RE may also be placed in the second deposition solution. In some embodiments, the second deposition solution can be composed of 5 mM tyramine in PBS. Using, for example, the external CE and/or the external RE, a CV can be used to reversibly scan the potential of the platinum black WE, while the needle sensor is submerged in the second deposition solution. In some embodiments, the range of the CV scan can be 0 to 1.4V at a scan rate of 10 mV/sec for 1 cycle. The CV scan can result in a polytramine membrane deposited over the absorbed GOx and HSA on the platinum black WE surface. The polytramine membrane may be glucose limiting.

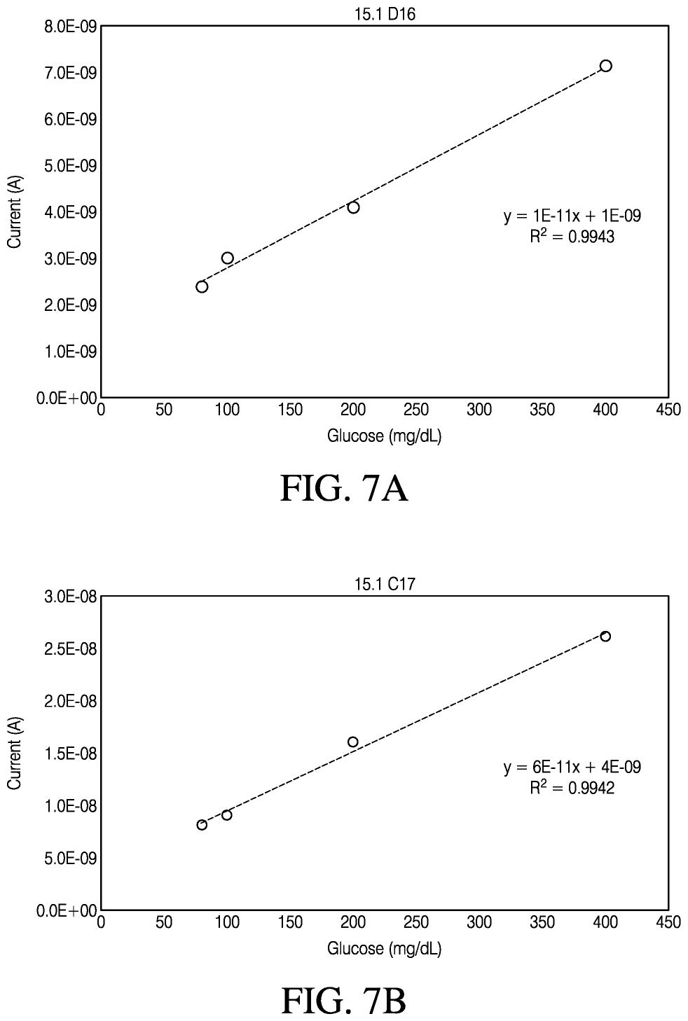

FIGS. 7A and 7B are additional examples of a linear response to glucose for sensors made according to a modified version of the method disclosed in FIG. 5 where the deposition solution also contains HSA stabilizer.

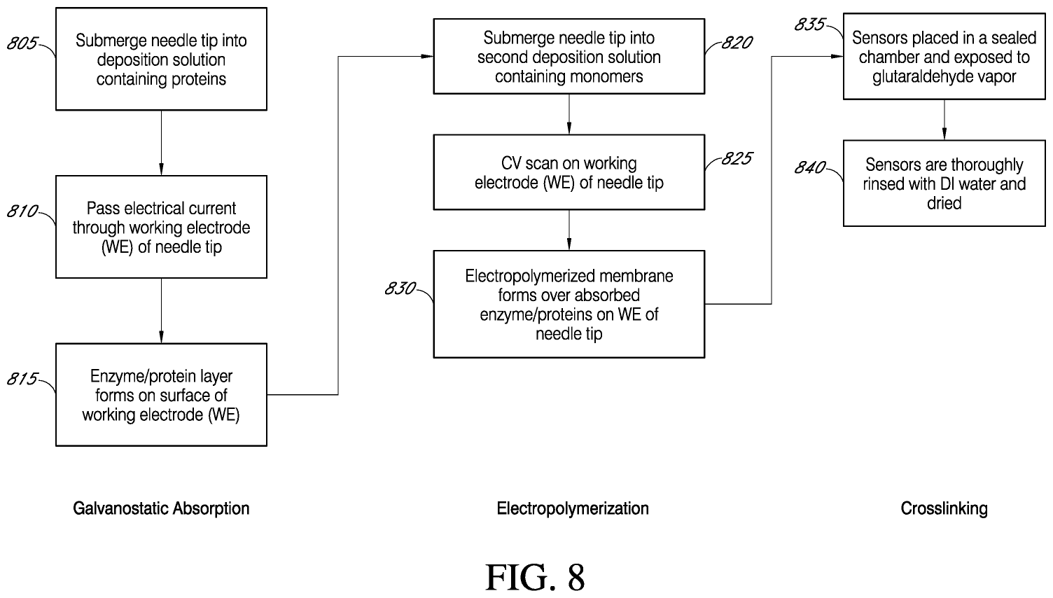

FIG. 8 is a flowchart showing a method for immobilizing an enzyme and diffusion limiting polymer on a working electrode of a biosensor needle tip, which includes stabilizing the enzyme matrix with glutaraldehyde crosslinking in accordance with example embodiments of the invention. The example here involves vapor phase crosslinking with glutaraldehyde. According to some example embodiments, the number and order of operations illustrated in FIG. 8 may vary. For example, according to some example embodiments, there may be fewer or additional operations, unless otherwise stated or implied to the contrary.

Referring to FIG. 8, GOx can be adsorbed on the surface of an evaporated Pt working electrode (WE) using galvanostatic adsorption. In this embodiment, the needle sensor can be submerged into a GOx/HSA deposition solution in step 805. An external CE and/or an external RE can also be placed in the deposition solution. In some embodiments, the GOx/HSA deposition solution can be composed of 4% wt/vol GOx and 4% wt/vol HSA in PBS. While the needle sensor is submerged in the GOx/HSA deposition solution, an electrical current can be passed through the evaporated Pt WE in step 810, for example, by utilizing the external CE and/or the external RE. In some embodiments, the current can be set at 1 μA and be held between 50 and 60 seconds. The electric current polarizes the evaporated Pt WE, which attracts the GOx and HSA proteins in the deposition solution to the evaporated Pt WE surface. The electrostatic forces hold the GOx and HSA at the surface of the evaporated Pt substrate in step 815.

Subsequently, a polymer membrane can be formed over the GOx on the surface of the evaporated Pt WE using cyclic voltammetry (CV) electropolymerization or fixed potential (CV/FP) electropolymerization. This process may involve removing the needle sensor from the initial GOx/HSA deposition solution and submerging it into a different container having a second deposition solution which contains monomers in step 820. The external CE and/or the external RE can also be placed in the second deposition solution. In some embodiments, the second deposition solution can be composed of 5 mM tyramine in PBS. In step 825, CV can be used to reversibly scan the potential of the evaporated Pt WE, while the needle sensor is submerged in the second deposition solution, for example, by utilizing the external CE and/or the external RE. In some embodiments, the range of the CV scan can be 0 to 1.2V at a scan rate of 10 mV/sec for 1 cycle. The CV scan can result in a polytramine membrane deposited over the absorbed GOx on the evaporated Pt WE surface in step 830. The polytramine membrane may be glucose limiting.

After drying, the needle sensor can be elevated in a sealed crosslinking chamber in step 835 for the case of vapor phase glutaraldehyde crosslinking. The crosslinking step can be performed before or after the electropolymerization of the membrane and can depend on the type of polymer membrane being used. In some embodiments, the crosslinking chamber may contain a volume of 5% glutaraldehyde in DI water and may be performed after electrodeposition of polytyramine. After crosslinking, the needle sensors can be thoroughly rinsed in DI water in step 840.

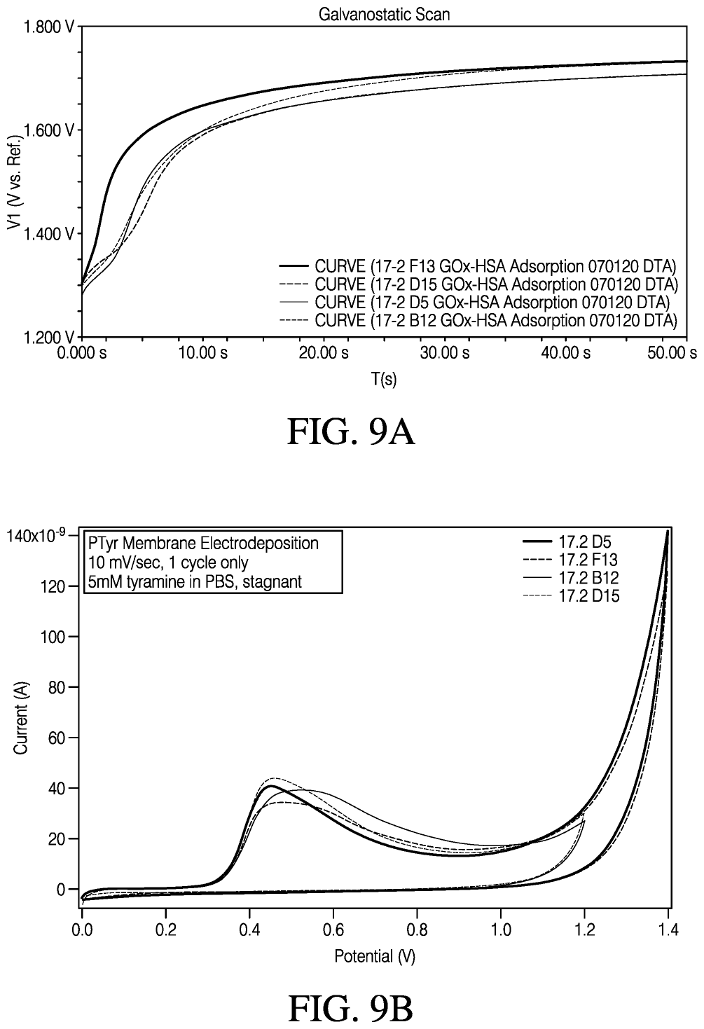

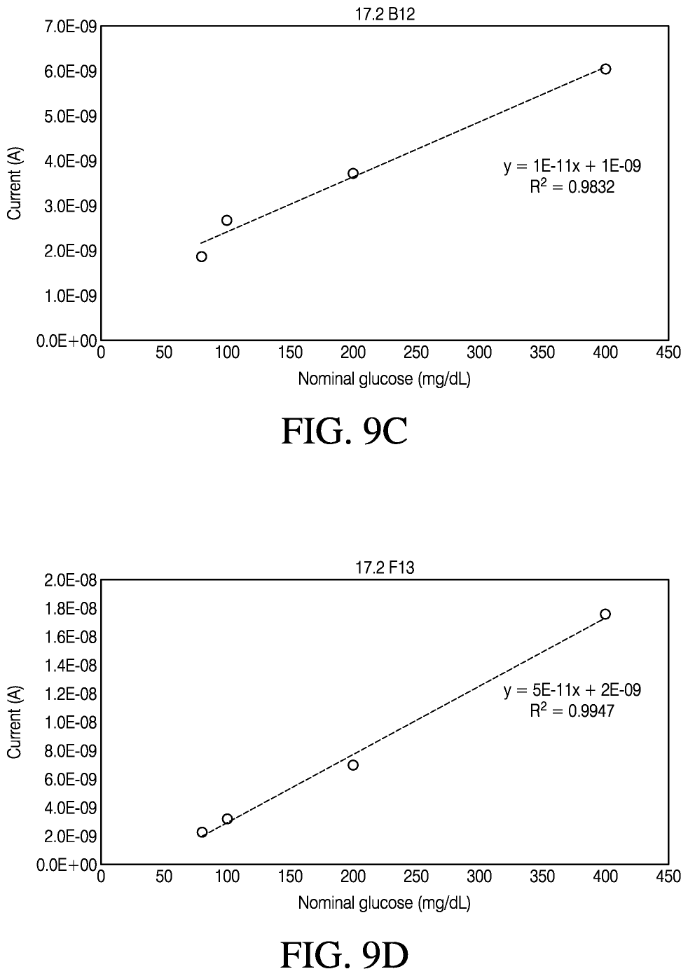

FIG. 9A shows the deposition trace for the galvanostatic absorption of enzyme onto the working electrode. FIG. 9B shows the cyclic voltammetry trace for the deposition of the diffusion limiting membrane which encapsulates the enzyme. Both the galvanostatic absorption of enzyme onto the working electrode in FIG. 9A and the deposition of the diffusion limiting membrane in FIG. 9B were performed according to the method disclosed in FIG. 5. FIGS. 9C and 9D are examples of a linear response to glucose for sensors made according to the method disclosed in FIG. 8, where the devices are crosslinked.

An alternative method for immobilizing an enzyme on an evaporated Pt biosensor electrode using a HSA deposition solution, in accordance with example embodiments of the disclosure, will now be described. According to some example embodiments, the number and order of operations described herein may also vary. For example, according to some example embodiments, there may be fewer or additional operations, unless otherwise stated or implied to the contrary.

In this alternative method, the needle sensor can be rinsed an additional time after the galvanostatic absorption and before the electropolymerization. Describing the entire alternative method in detail, the needle sensor can first be submerged into a GOx/HSA deposition solution with an external CE and/or an external RE. In some embodiments, the GOx/HSA deposition solution can be composed of 4% wt/vol GOx and 4% wt/vol HSA in PBS. An electrical current can then be passed through the evaporated Pt WE while the needle sensor is submerged in the GOx/HSA deposition solution, for example, by utilizing the external CE and/or the external RE. In some embodiments, the current can be set at 1 μA and be held between 50 and 60 seconds. The electric current polarizes the evaporated Pt WE, which attracts the GOx and HSA in the deposition solution to the evaporated Pt WE surface. The electrostatic forces hold the GOx and HSA at the surface of the evaporated Pt WE.

Subsequently, a polymer membrane can be formed over the GOx on the surface of the evaporated Pt WE using cyclic voltammetry (CV) electropolymerization. This process involves removing the evaporated needle sensor from the initial GOx/HSA deposition solution and submerging the needle sensor into a different container having a second deposition solution containing monomers. The external CE and/or the external RE can also be placed in the second deposition solution. In some embodiments, the second deposition solution can be composed of 5 mM tyramine in PBS. Using, for example, the external CE and/or the external RE, a CV can be used to change the potential of the evaporated Pt WE, while the needle sensor is submerged in the second deposition solution. In some embodiments, the range of the CV scan can be 0 to 1.2V at a scan rate of 10 mV/sec for 1 cycle. The CV scan can result in a polytramine membrane deposited over the absorbed GOx and HSA on the evaporated Pt WE surface. The polytramine membrane may be glucose limiting.

After drying, the needle sensor can be elevated in a sealed crosslinking chamber for the case of vapor phase glutaraldehyde crosslinking. The crosslinking step can be performed before or after the electropolymerization of the membrane and can depend on the type of polymer membrane being used. In some embodiments, the crosslinking chamber may contain a volume of 5% glutaraldehyde in DI water and may be performed after electrodeposition of polytyramine. After crosslinking, the needle sensors can be thoroughly rinsed in DI water.

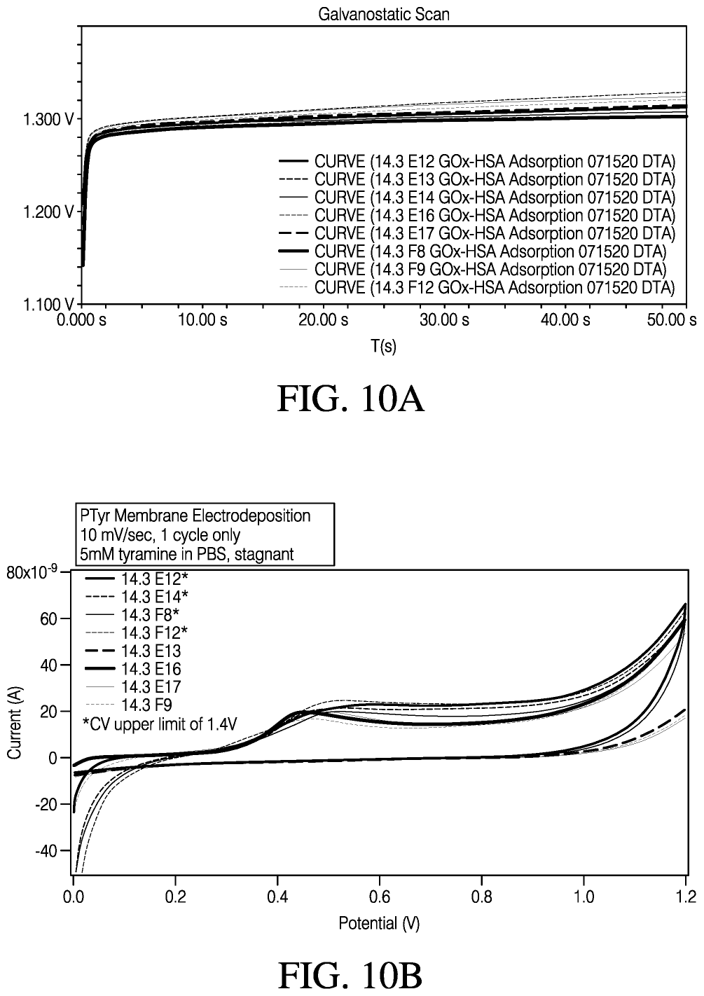

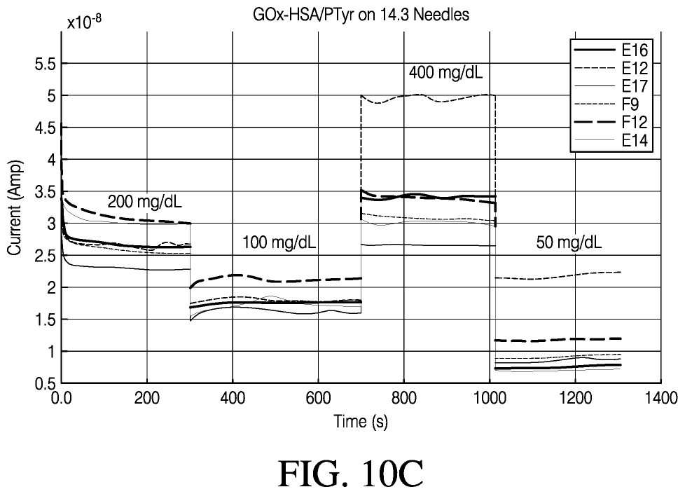

FIG. 10A shows the deposition trace for the galvanostatic absorption of enzyme onto the working electrode according to the method disclosed in FIG. 5. FIG. 10B shows the cyclic voltammetry trace for the deposition of the diffusion limiting membrane which encapsulates the enzyme according to the method disclosed in FIG. 5. FIG. 10C shows the glucose response of a set of sensors that has been fabricated according to the method disclosed in FIG. 8.

In addition to the above-described electrochemical methods for selectively depositing enzymes, various photochemical deposition technologies can also be used to selectively deposit enzymes on the working electrodes of the sensors. Some advantages of using photochemical based deposition techniques may include speed (e.g., being virtually instantaneous), less or no dependence on temperature and pH, and lower or no probability of decreasing biochemical activity of the enzyme deposited on the working electrode surface.

The simplest approach to photo-enhanced deposition uses a traditional photoresist. In this process, a photoresist, preferably a positive photo resist, is used to create a window over the working electrode. An enzyme (e.g., GOx) or an enzyme added with a protein is deposited over the entire wafer. The enzyme can then be crosslinked, and the photoresist layer can be removed using a lift-off technique. A solvent should be used for the lift-off technique that does not impact the stability of the enzyme. This process should result in the crosslinked enzyme deposited only over the working electrode.

In other embodiments, the photochemical deposition technique may involve depositing an enzymatic sensing layer in a photocured polymer system. A photocured polymer (e.g., photopolymer) may include a polymer that changes its physical properties when cured (e.g., when exposed to light such as ultraviolet (UV) light). For example, the photocured polymer may harden as a result of crosslinking within the polymer when exposed to the UV light.

Some example photocured polymers may include, but are not limited to, polyurethanes, acrylates, polyvinyl alcohol (PVA) based polymers, styrene-based polymers, photoresists, epoxies, and other photocurable polymers.

Additionally, the photocured polymer system may include a photocured hydrogel system. A hydrogel is a network of cross-linked polymer chains that are hydrophilic. Some examples of photocured hydrogel systems may include, but are not limited to, polyethylene glycol diacrylate (PEGDA), methacrylated gelatin (GELMA), methacrylated hyaluronic acid (MeHA), methacrylated alginate-based hydrogels as well as methacrylated versions of polyethylene glycol (PEG), PVA, chondroitin, chitosan, dextran and other alginates.

Some approaches for creating a photocured hydrogel system may include free-radical polymerization that uses a photoinitiator (e.g., a molecule that creates reactive specifies such as free radicals, cations or anions when exposed to radiation such as UV light) and photodimerization. Some example photoinitiators may include, but are not limited to, 2,2-Dimethoxy-2-phenylacetophenone (DMPA), benzophenones, thioxanthones, and other known photoinitiators.

According to various embodiments, the process for depositing the enzyme on the working electrode using photochemical deposition techniques can begin by preparing an aqueous solution containing the enzyme and photocurable components. The photocurable components may include, but are not limited to, monomers (e.g photocurable monomers), a photoinitiator, and crosslinkers. In some embodiments, human serum albumin (HSA) can also be incorporated in the aqueous solution as a stabilizing protein for GOx, when GOx is used as the enzyme, but other stabilizing proteins such as silk fibroin may be incorporated. Some example photocurable monomers may include hydroxyethylmethacrylate (HEMA), polyethylene glycol (PEG), and others. Some example crosslinkers may include any di-acrylate based monomers (e.g., N—N′ diallyltartardiamide (DATD), piperazine di-acrylamide (PDA), N,N′-bis(acryloyl)cystamine (BAC) diethylene glycol diacrylate, polyethylene glycol diacrylate (PEGDA), 2,6-pyridinediethanol dimethacrylate, polyethyleneoxide diacrylate, and many others). In one embodiment, PEGDA can be used as a crosslinker. Some example photoinitiators may include DMPA, benzophenones, thioxanthones, and other known photoinitiators.

The resulting aqueous solution can be deposited on the sensor via any suitable method including, but not limited to, micro-dispense, spin coating, spray coating and dip coating. After depositing the aqueous solution on the sensor, UV light can be illuminated on the working electrode only, which creates a localized cured hydrogel. After photo curing the non-photo-linked material is simply washed off.

Several methods can be used to localize the illumination on a sensor or on a wafer containing a multitude of sensors. Some of these methods may include traditional photoresists, shadow masks, or fiber-optic based delivery systems. In a fiber-optic based systems, an x-y stage can be used to move either the illumination fiber over the wafer or move the wafer under the illumination fiber. Light sources may include, but not limited to, a UV bulb, an LED, or a Laser Diode based.

In some embodiments, the enzyme deposited on the sensor can be subsequently crosslinked using glutaraldehyde or any other bifunctional protein crosslinker after creating the hydrogel.

In some embodiments, the enzyme may be functionalized initially before added to the aqueous solution, which may allow it to be photocrosslinked inside the hydrogel. This approach can reduce or remove a need for any additional crosslinking. To functionalize the enzyme there are a few well understood approaches including reacting the enzyme with acrylate-PEG-n-hydroxysuccinimide (PEG-NHS) or by conjugation to polyacrylic acid. For example, the enzyme may be stirred with the PEG-NHS or with the polyacrylic acid in solution.

One advantage of using the acrylate PEG-NHS system is that it allows for tailoring the resulting hydrogel more easily. Some PEG-NHS systems can range from zero PEG to PEG 2000, to PEG 5000. In some embodiments, blends of the PEG percentages and hydrogels with the required physical properties can be used.

In some embodiments, an analyte (e.g., glucose) limiting membrane can be added on top of the photochemically deposited enzyme using electropolymerization techniques. As discussed previously, the electropolymerization technique is a process for creating polymer films on an electrode by controlling the voltage or current that is applied to the electrode while immersed in an electrolyte containing monomeric species (e.g., a monomer that can be polymerized by applying an appropriate voltage/current, such monomers are electro-polymerizable). Several electrochemical approaches can be used to create the electropolymerized polymers. These approaches may include constant voltage, constant current, cyclic voltammetry, unbalanced AC as well as a series of pulses with constant or varying voltages and times.

In one embodiment, cyclic voltammetry (CV) can be used to create the analyte limiting membrane over the photochemically deposited enzyme. According to this embodiment, the sensor with the photochemically deposited enzyme can be immersed in a deposition solution. The potential of the working electrode is scanned over a specific range and cycled such that the resulting current can be measured. For example, a potential scan can begin from a greater potential and end at a lower potential. The scan can then cycle from the lower potential back to the greater potential. The current created by the cycle changes can create a polymer film on the surface of the working electrode due to oxidation or reduction of the monomeric species in the deposition solution for this process.

In other embodiments, the analyte limiting membrane may be added over the deposited enzyme using photochemical techniques. For example, the analyte limiting membrane by may be added over the deposited enzyme layer using micro-dispense, spin coating, spray coating, and other suitable deposition techniques. Subsequently, the analyte limiting membrane may be cured using photoillumination. For example, the analyte limiting membrane may include photocured polyvinyl alcohol (PVA) created by the photoillumination of a Poly(vinyl alcohol), N-methyl-4(4′-formylstyryl)pyridinium methosulfate acetal (PVA-SBQ) polymer. In addition to PVA, various other hydrogel based systems may also be used for the analyte limiting membrane.

In some embodiments, the photochemical chemical techniques described above can also be used to add an analyte limiting membrane over an enzyme that has been deposited using electrochemical techniques (e.g., galvanostatic adsorption). For example, the analyte limiting membrane by may be added over the electrochemically deposited enzyme layer using micro-dispense, spin coating, spray coating, and other suitable deposition techniques. Subsequently, the analyte limiting membrane may be cured using photoillumination.

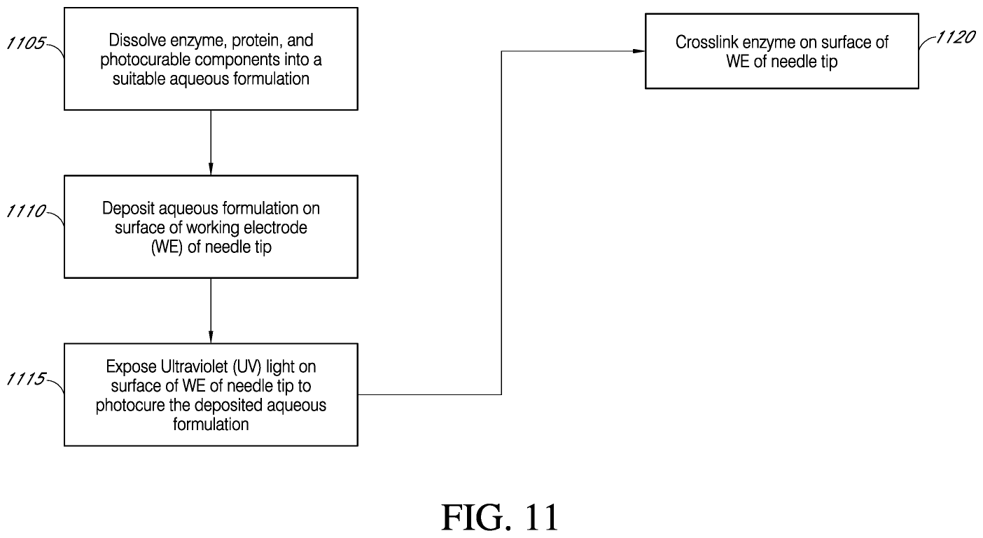

FIG. 11 is a flowchart showing a method for immobilizing an enzyme on a working electrode of a biosensor needle tip using photochemical techniques, in accordance with example embodiments of the invention. According to some example embodiments, the number and order of operations illustrated in FIG. 11 may vary. For example, according to some example embodiments, there may be fewer or additional operations, unless otherwise stated or implied to the contrary.

Referring to FIG. 11, the method can begin by preparing an aqueous solution containing an enzyme (e.g., GOx) and photocurable components in 1105. The photocurable components may include, but are not limited to, monomers (e.g photocurable monomers), a photoinitiator, and crosslinkers. In some embodiments, HSA can also be incorporated in the aqueous solution as a stabilizing protein for GOx but other stabilizing proteins such as silk fibroin may be incorporated. In some embodiments, the photocurable monomers may include HEMA, PEG, or other photocurable monomers. In some embodiments, the crosslinkers may be include any di-acrylate based monomers (e.g., DATD, PDA, BAC (dallyltartardiamine, piperazine diacrylate, bisacrylcystamine), diethylene glycol diacrylate, polyethylene glycol diacrylate, 2,6-pyridinediethanol dimethacrylate, polyethyleneoxide diacrylate, and many others). In some embodiments, the photoinitiators may include DMPA, benzophenones, thioxanthones, or other known photoinitiators.

Various amounts of the components of the aqueous solutions can be used. In some embodiments, 5-95% of the aqueous solution may include photocurable monomers. In some embodiments, 0.1-10% of the aqueous solution may include photoinitiators. In some embodiments, 0-50% of the aqueous solution may include crosslinkers. In some embodiments, 0.1-10% of the aqueous solution may include enzymes. In some embodiments, 0.1-20% may include HSA. The ratios of the components of the aqueous solution can be optimized on a case-by-case basis.

The resulting aqueous solution can be deposited on the surface of the working electrode of the biosensor needle tip in 1110. In some embodiments, the aqueous solution can be deposited using micro-dispense, spin coating, spray coating, and other suitable deposition techniques. After depositing the aqueous solution on the working electrode, the surface of the working electrode can be illuminated by UV light in 1115. The UV light can cure the deposited aqueous formulation to form a hydrogel. In some embodiments, the UV light may be a UV bulb, LED, Laser diode or other UV light source. In some embodiments, the UV light illumination can be localized using traditional photoresists, shadow masks, and fiber-optic based delivery systems

After the hydrogel is formed, the enzyme can be cross-linked in 1120. In some embodiments, the enzyme can be cross-linked using a bifunctional protein crosslinker. In some embodiments, enzymes may be cross-linked with glutaraldehyde or one of various carbodiimides.

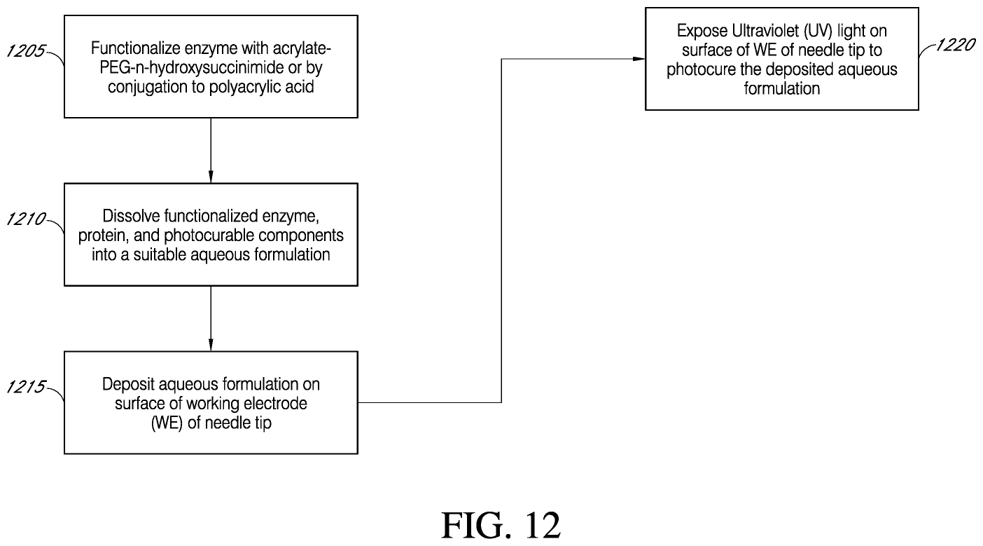

FIG. 12 is a flowchart showing a method for immobilizing an enzyme on a working electrode of a biosensor needle tip using a functionalized enzyme, in accordance with example embodiments of the invention. According to some example embodiments, the number and order of operations illustrated in FIG. 12 may vary. For example, according to some example embodiments, there may be fewer or additional operations, unless otherwise stated or implied to the contrary.

Referring to FIG. 12, the enzyme (e.g., GOx) can be functionalized in 1205 before adding to an aqueous solution. In some embodiments, the GOx can be functionalized with PEG-n-hydroxysuccinimide or by conjugation to polyacrylic acid.

After functionalizing the enzyme, an aqueous solution containing the functionalized enzyme and photocurable components can be prepared in 1210. The photocurable components may include, but are not limited to, monomers (e.g photocurable monomers), a photoinitiator, and crosslinkers. In some embodiments, HSA can also be incorporated in the aqueous solution as a stabilizing protein for GOx but other stabilizing proteins such as silk fibroin may be incorporated. In some embodiments, the photocurable monomers may include HEMA, PEG, or other photocurable monomers. In some embodiments, the crosslinkers may be include any di-acrylate based monomers (e.g., DATD, PDA, BAC (dallyltartardiamine, piperazine diacrylate, bisacrylcystamine), diethylene glycol diacrylate, polyethylene glycol diacrylate, 2,6-pyridinediethanol dimethacrylate, polyethyleneoxide diacrylate, and many others). In some embodiments, the photoinitiators may include DMPA, benzophenones, thioxanthones, or other known photoinitiators.

The resulting aqueous solution can be deposited on the surface of the working electrode of the biosensor needle tip in 1215. In some embodiments, the aqueous solution can be deposited using micro-dispense, spin coating, spray coating, and other suitable deposition techniques. After depositing the aqueous solution on the working electrode, the surface of the working electrode can be illuminated by UV light in 1220. The UV light can cure the deposited aqueous formulation to form a hydrogel. In some embodiments, the UV light may be a UV bulb, LED, Laser diode or other UV light source. In some embodiments, the UV light illumination can be localized using traditional photoresists, shadow masks, and fiber-optic based delivery systems

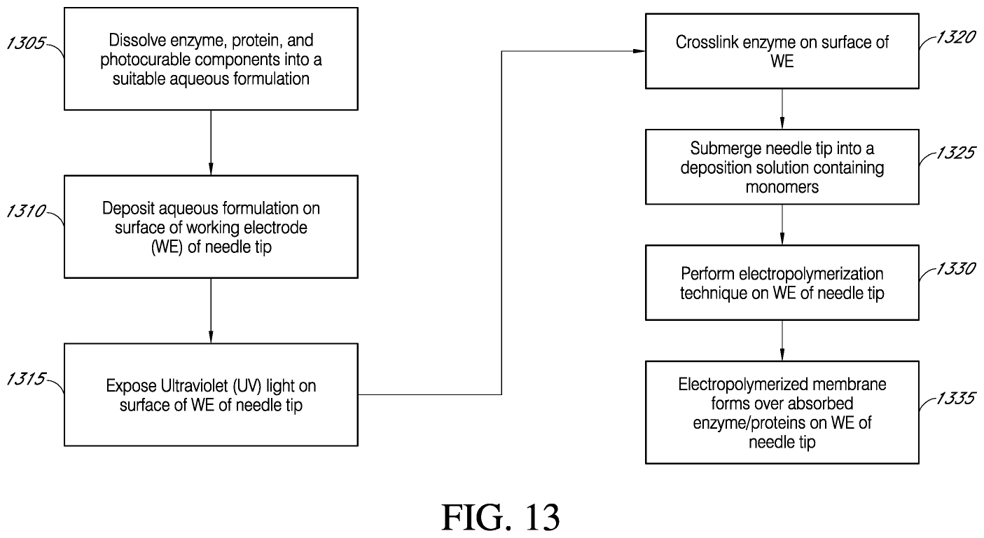

FIG. 13 is a flowchart showing a method for immobilizing an enzyme on a working electrode of a biosensor needle tip and adding an electropolymerized membrane over the absorbed enzyme, in accordance with example embodiments of the invention. According to some example embodiments, the number and order of operations illustrated in FIG. 13 may vary. For example, according to some example embodiments, there may be fewer or additional operations, unless otherwise stated or implied to the contrary.

Referring to FIG. 13, the method can begin by preparing an aqueous solution containing an enzyme (e.g., GOx) and photocurable components in 1305. The photocurable components may include, but are not limited to, monomers (e.g photocurable monomers), a photoinitiator, and crosslinkers. In some embodiments, HSA can also be incorporated in the aqueous solution as a stabilizing protein for the GOx but other stabilizing proteins such as silk fibroin may be incorporated. In some embodiments, the photocurable monomers may include HEMA, PEG, or other photocurable monomers. In some embodiments, the crosslinkers may be include any di-acrylate based monomers (e.g., DATD, PDA, BAC (dallyltartardiamine, piperazine diacrylate, bisacrylcystamine), diethylene glycol diacrylate, polyethylene glycol diacrylate, 2,6-pyridinediethanol dimethacrylate, polyethyleneoxide diacrylate, and many others). In some embodiments, the photoinitiators may include DMPA, benzophenones, thioxanthones, or other known photoinitiators.

The resulting aqueous solution can be deposited on the surface of the working electrode of the biosensor needle tip in 1310. In some embodiments, the aqueous solution can be deposited using micro-dispense, spin coating, spray coating, and other suitable deposition techniques. After depositing the aqueous solution on the working electrode, the surface of the working electrode can be illuminated by ultraviolet (UV) light in 1315. The UV light can cure the deposited aqueous formulation to form a hydrogel. In some embodiments, the UV light may be a UV bulb, LED, Laser diode or other UV light source. In some embodiments, the UV light illumination can be localized using traditional photoresists, shadow masks, and fiber-optic based delivery systems

After the hydrogel is formed, the enzyme can be cross-linked in 1320. In some embodiments, the enzyme can be cross-linked using a difunctional protein crosslinker.

Subsequently, a polymer membrane can be formed over the GOx and associated proteins on the surface of the WE using electropolymerization techniques. Some example electropolymerization techniques may include cyclic voltammetry or fixed potential (CV/FP) electropolymerization. In 1325, the needle tip with the crosslinked enzyme can be submerged into a deposition solution. In some embodiments, the deposition solution may contain monomers (e.g., electro-polymerizable monomers). The external CE and/or the external RE can also be placed in the deposition solution. In some embodiments, the second deposition solution can be composed of 5 mM tyramine in PBS. In step 1330, electropolymerization techniques can be applied to the WE of the needle tip. In one embodiment, CV can be used to reversibly scan the potential of the WE, for example, by utilizing the external CE and/or the external RE. In this embodiment, the range of the CV scan can be 0 to 1.4V at a scan rate of 10 mV/sec for 1 cycle. The electropolymerization can result in a polytramine membrane deposited over the absorbed GOx on the WE in step 1335. The polytramine membrane may be glucose limiting.

The foregoing is illustrative of example embodiments, and is not to be construed as limiting thereof. Although a few example embodiments have been described, those skilled in the art will readily appreciate that many modifications are possible in the example embodiments without materially departing from the novel teachings and advantages of example embodiments. Accordingly, all such modifications are intended to be included within the scope of example embodiments as defined in the claims. Therefore, it is to be understood that the foregoing is illustrative of example embodiments and is not to be construed as limited to the specific embodiments disclosed, and that modifications to the disclosed example embodiments, as well as other example embodiments, are intended to be included within the scope of the appended claims. The inventive concept is defined by the following claims, with equivalents of the claims to be included therein.

1. A method for forming an enzymatic biosensor comprising an enzyme configured to catalyze the oxidation of glucose and three or fewer electrodes configured to directly measure an amount of a product formed from the oxidation of the glucose, the method comprising:
preparing an aqueous solution comprising the enzyme and photocurable components;
depositing the aqueous solution on a surface of a working electrode of a substrate from among the three or fewer electrodes;
illuminating the working electrode with ultraviolet (UV) light to cure the aqueous solution; and
crosslinking the enzyme deposited on the working electrode with solution phase or vapor phase crosslinking after curing the aqueous solution.

2. The method of claim 1, the method further comprising:
placing the substrate in a deposition solution comprising electro-polymerizable monomers; and
passing a current through the working electrode to polymerize the monomers to form an electropolymerized polymer layer over the enzyme deposited on the working electrode.

3. The method of claim 2, wherein the deposition solution comprise comprises 5 mM tyramine in phosphate-buffered saline (PBS).

4. The method of claim 2, wherein the electropolymerized polymer layer comprise a polytramine membrane.

5. The method of claim 1, wherein the photocurable components comprise at least one of a photocurable monomer, a photoinitiator, and a crosslinker.

6. The method of claim 5, wherein the aqueous solution further comprises a stabilizing protein.

7. The method of claim 6, wherein the stabilizing protein comprises human serum albumin (HSA).

8. The method of claim 6, wherein the stabilizing protein comprises silk fibroin.

9. The method of claim 1, wherein the enzyme comprises glucose oxidase (GOx).

10. The method of claim 1, wherein the working electrode comprises at least one of platinum, platinum black, carbon, iridium oxide, or platinum nanoparticles.

11. A method for forming an enzymatic biosensor comprising an enzyme configured to catalyze the oxidation of glucose and three or fewer electrodes configured to directly measure an amount of a product formed from the oxidation of the glucose, the method comprising:
functionalizing the enzyme;
preparing an aqueous solution comprising the enzyme and photocurable components;
depositing the aqueous solution on a surface of a working electrode of a substrate from among the three or fewer electrodes; and
illuminating the working electrode with ultraviolet (UV) light to cure the aqueous solution.

12. The method of claim 11, wherein the enzyme is functionalized by reacting the enzyme with acrylate-PEG-n-hydroxysuccinimide.

13. The method of claim 11, wherein the enzyme is functionalized by reacting the enzyme with polyacrylic acid.

14. The method of claim 11, wherein the photocurable components comprise at least one of a photocurable monomer, a photoinitiator, and a crosslinker.

15. The method of claim 14, wherein the aqueous solution further comprises a stabilizing protein.

16. The method of claim 15, wherein the stabilizing protein comprises human serum albumin (HSA).

17. The method of claim 15, wherein the stabilizing protein comprises silk fibroin.

18. The method of claim 11, wherein the enzyme comprises glucose oxidase (GOx).

19. The method of claim 11, wherein the working electrode comprises at least one of platinum, platinum black, carbon, iridium oxide, or platinum nanoparticles.

20. A method for forming an enzymatic biosensor comprising an enzyme configured to catalyze the oxidation of glucose and three or fewer electrodes configured to directly measure an amount of a product formed from the oxidation of the glucose, the method comprising:
providing the enzymatic biosensor on a rigid substrate;
depositing the enzyme on a working electrode of the rigid substrate from among the three or fewer electrodes; and
electropolymerizing polymer on the enzyme deposited on the working electrode,
wherein depositing the enzyme on the working electrode comprises preparing an aqueous solution that comprises the enzyme and photocurable components, depositing the aqueous solution on the working electrode, exposing ultraviolet (UV) light on a surface of the working electrode to cure the aqueous solution on the surface of the working electrode, and crosslinking the enzyme deposited on the working electrode with solution phase or vapor phase crosslinking after curing the aqueous solution.

21. The method of claim 1, wherein only portions of the working electrode are illuminated with UV light, to cure the aqueous solution on the surface of the working electrode without curing the aqueous solution in areas other than the working electrode.

22. The method of claim 1, wherein the product configured to be measured by the working electrode comprises hydrogen peroxide.

23. The method of claim 1, wherein the measurement of the amount of the product formed from the oxidation of the glucose is measured independently from measurements derived from any electrodes other than the three or fewer electrodes.

24. The method of claim 1, wherein a region of the enzymatic biosensor that directly interacts with the glucose is devoid of any transistors.
